(19) United States
(12) Patent Application Publication
(10) Pub. No.: US 2009/0227038 A1
(43) Pub. Date: Sep. 10, 2009

(54) METHODS OF DETERMINING SKELETAL MATURITY

(76) Inventor: Mohamed I. Masoud, Boston, MA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/296,086

(22) PCT Filed: Apr. 5, 2007
(86) PCT No.: PCT/US07/08532

§ 371 Date: May 8, 2009

Related U.S. Application Data
(63) Continuation-in-part of application No. 60/790,302, filed on Apr. 6, 2006.

(51) Int. Cl. G01N 33/74

(52) U.S. Cl. 436/87

(57) ABSTRACT

The present invention provides methods to determine skeletal maturity.

### BACKGROUND

The efficacy of many medical procedures, including orthodontic treatment, orthognathic surgery, and dental implantology, is highly dependent the determination of skeletal maturity in patients. Residual bone growth may interfere with certain procedures, while other procedures work best in patients who are still experiencing bone growth. Accordingly, the accurate determination of skeletal maturity plays a key role in the timing of such medical procedures. One method to evaluate skeletal maturity involves X-rays. In addition to radiographic exposure and the subjectivity of staging x-rays however, a major disadvantage of hand wrist radiographs and cervical vertebral staging is that the final stage of development does not necessarily indicate the completion of growth, especially mandibular growth. Several studies have shown that mandibular growth continues after radiographic skeletal maturity. Accordingly, better methods are needed to determine skeletal maturity.

### SUMMARY OF THE INVENTION

The invention provides methods to determine skeletal maturity in a mammal (e.g., human). These methods are useful to determine our appropriate time to perform a medical procedure on a mammal including, for example, orthodontic procedures, surgical procedures, or tooth implants.

Accordingly, a method for determining skeletal maturity in a subject, is carried out by measuring the level of IGF-1 in a tissue or fluid sample. An elevated level of IGF-1 post-puberty indicates that the subject has not yet reached skeletal maturity. For example, the level of IGF-1 exceeds 250 μg/L serum in a post-pubertal subject. A reduced level of IGF-1 (e.g., less than 250 μg/L) pre-puberty indicates that the subject has not yet undergone the growth spurt associated with puberty. Elevated levels of IGF-1 (e.g., greater than 250 μg/L, e.g., 300-400 μg/L) indicates that the subject is in puberty and undergoing a pubertal growth spurt. In certain medical procedures (e.g., treatment involving the use of a protraction headgear to correct skeletal abnormalities associated with skeletal dental class III, i.e., those having a large lower jaw), patients may need to be treated before undergoing a pubertal growth spurt. Such pre-pubertal patients are distinguished from patients in early puberty by having IGF-1 levels that are less than 250 μg/L. Certain procedures however, work best during bone growth (e.g., pubertal bone growth and residual bone growth) and therefore patients who are undergoing pubertal growth spurt are preferred candidates. Such patients are identified as having IGF-1 levels that are greater than 250 μg/L. These procedures include those that correct skeletal abnormalities that are associated with skeletal class II (small lower jaw). The use of traditional X-rays sometimes fail to identify growth spurts because X-rays are limited in their use (e.g., radiation exposure). Furthermore, X-rays fail to recognize the intensity of growth spurts. Various other procedures work optimally when bone growth has ended. The level of IGF-1 in such patients is less than 250 μg/L. This method is particularly useful for procedures involving facial bones since facial growth lags behind statural growth. Accordingly, patients whose height has stabilized may still be experiencing facial growth (since facial growth lags behind statural growth) and are therefore not ideal candidates for procedures that work best in the absence of residual bone growth. One exemplary procedure is a surgical procedure that corrects skeletal abnormalities associated with skeletal dental class III.

In this invention, a post-pubertal subject is identified using the Tanner scale, cervical staging (a post-pubertal subject being at cervical stage 6), chronological age (14.5 years old for females, 17.5 years old for males), height stabilization, sexual maturity (e.g., breast size, testicular size, penis size, menstruation, or presence of pubic hair), hand-wrist radiograph, measurements of bone growth over time, or other known methods of determining the boundaries of puberty (Marshall et al., Arch Dis Child, 1969). Preferably, the method does not comprise X-ray examination of a cervical vertebra. The non-radiological methods of identifying skeletal maturation and/or mandibular/facial bone growth maturation determine the time at which diagnosis and treatment of skeletal disorders and dental orthodontal manipulations take place. The methods of the invention are independent of hyoid bone, cervical vertebrae, hand bones, testicular size, body mass index, standing height determinations, or other means of pubertal age determinations.

In one method, a biological sample is obtained from a mammal and IGF-1 levels are measured. IGF-1 levels ranging between 120 and 280 ug/L, 130 and 270 ug/L, 140 and 260 ug/L, 150 and 250 ug/L, or 160 and 250 ug/L indicate that a mammal in prepubertal stage is at CS1 or CS2, levels ranging between 250 and 330 ug/L, 260 and 330 ug/L, 270 and 320 ug/L, or 280 and 320 ug/L indicate that a mammal in early pubertal stage is at CS3, levels ranging between 310 and 400 ug/L, 320 and 390 ug/L, 320 and 380 ug/L, or 330 and 380 ug/L indicate that a mammal in pubertal stage is at CS4, levels ranging between 350 and 600 ug/L, 360 and 550 ug/L, 380 and 500 ug/L, 390 and 500 ug/L, or 400 and 500 ug/L indicate that a mammal in late pubertal stage is at CS5, and levels ranging between 200 and 450 ug/L, 200 and 400 ug/L, or 250 ug/L indicate that a mammal in post pubertal stage is at CS6. Pubertal stage is determined based on chronological age or sexual characteristics of the mammal.

Using the information obtained as described above, the proper timing for a medical or dental procedure is determined. For example, a mammal that is identified as being at CS6 and has IGF-1 that are less than 300 ug/L, 290 ug/L, 280 ug/L, 270 ug/L, 260 ug/L, 250 ug/L, 230 ug/L, or 200 ug/L, is one that has gone through its growth spurt. Since there should not be any residual bone growth, this mammal is a candidate for a medical procedure where residual bone growth is undesirable. Alternatively, a mammal whose IGF-1 levels range between 260 and 500 ug/L and who is identified as being at CS1, CS2, CS3, CS4, or CS5 is a candidate for a medical procedure that involves or requires residual bone growth. Alternatively, a mammal having IGF-1 levels below 280 ug/L and identified as being at CS6 is a candidate for a medical procedure that does not involve residual bone growth.

Alternatively, samples are collected periodically from a mammal (e.g., every two years, every year, every six months, or every three months) and the levels of IGF-1 in the samples are measured over time. The skeletal maturity stage of the mammal is determined at each time point. An increase in IGF-1 levels over time indicates that the mammal is between cervical stage CS1 and CS5 and a reduction of IGF-1 levels over time indicates that the mammal is between CS5 and CS6. For example, a mammal whose IGF-1 levels are increasing over time and is identified as being at CS1, CS2, CS3, CS4, or CS5 is selected as a candidate for a medical procedure that involves residual bone growth. Desirably, the levels of IGF-1 are greater than 150 ug/L, 200 ug/L, or 250 ug/L. For procedures in which bone growth should be complete (i.e., no residual bone growth expected), a candidate mammal is one having been identified as being at CS6 and whose IGF-1 levels are less than 300 ug/L, 290 ug/L, 280 ug/L, or 250 ug/L. Samples may be collected at anytime i.e., pre-pubertal stage, pubertal stage, late pubertal stage, and post-pubertal stage.

In all foregoing aspects of the invention, a biological sample is a blood sample, a urine sample, saliva sample, or a serum sample. Blood samples may be obtained by blood spotting. The methods may be used to determine facial bone growth (e.g., mandibullar growth). If desired, a hand-wrist radiograph is also performed and is evaluated using the Greulich and Pyle technique or the Fishman technique. Pubertal stages such as the pre-pubertal stage, pubertal stage, late pubertal stage, and post-pubertal stage are identified by physical characteristics (e.g., sexual characteristics) of the mammal or using the chronological age of the mammal. Puberty typically occurs in girls that are between 11 and 13 years old and occurs in boys between 13 and 16. The pubertal growth spurt occurs in girls aged between 9.5 and 14.5 years old and in boys aged between 10.5 and 16 or 13 and 17.5 years old. In girls, pubic hair appears between the ages of 8 and 14, menarche occurs between the age of 10 and 16.5, and breast development from the time of budding occurs between the ages of 8 and 13. In boys, the penis size change occurs between the ages of 11 and 14.5 or 13.5 and 17, testicular growth occurs between the age of 10 and 13.5 or between 14.5 and 18, and the appearance of pubic hair occurs between the ages of 10 and 15 or 14 and 18.

### BRIEF DESCRIPTION OF THE FIGURES

FIG. 1 is a series of radiographs showing reference examples of the six cervical stages.

FIG. 2 is a graph showing mean IGF-1 levels and the 95% confidence interval for each cervical stage.

FIG. 3 is a series of radiographs showing the effect of positional changes of the same vertebra on its radiographic appearance.

### DETAILED DESCRIPTION

Accurate determination of skeletal maturity and residual growth is crucial to many orthodontic, orthognathic, and dental implant timing decisions. The invention is based on the discovery that IGF-1, a mediator for growth hormone, is a reliable marker of skeletal maturity particularly the terminus of facial bone growth.

Samples were collected from 83 patients in a cross-sectional study. Height and weight measurements, a lateral cephalometric radiograph, and a blood spot sample were collected from each patient on the same day. Patients were either on recall to begin orthodontic treatment, in active treatment, or in post-treatment follow up. The correlation between IGF-1 levels and skeletal maturity was determined by the cervical vertebra on the lateral cephalometric radiographs. One-way ANOVA analysis showed that the mean IGF-1 levels were significantly higher in the late pubertal stages compared to the prepubertal, early pubertal, and post pubertal stages. Linear correlation showed that IGF-1 levels have a significant positive correlation with skeletal maturity as determined by radiographs from the prepubertal stages to the late pubertal stages. Linear correlation from the late pubertal stages to the post pubertal stage showed a significant negative correlation with IGF-1 levels approaching prepubertal levels. Within the postpubertal stage, IGF-1 levels had a negative linear correlation with increasing time from the onset of puberty, as well as chronological age. Thus, there was a statistically significant difference between the mean blood-spot IGF-1 levels of individuals in their pubertal growth spurt according to hand wrist and cervical vertebral stages and the mean IGF-1 of individuals in their pre and post pubertal stages according to hand wrist and cervical vertebral stages. The results indicate that measuring levels of IGF-1 using blood spotting is a reliable marker for skeletal maturity, particularly to detect residual growth in young adults. Accordingly, the invention provides methods to evaluate skeletal maturity in a mammal by obtaining a blood spot from a mammal and determining its IGF-1 levels. This technique is particularly useful given that blood spotting is a non-invasive technique. In the studies described herein, blood spot samples were collected using painless lancets with no visible needles.

The determination of skeletal age is instrumental for the design and timing of various treatments of skeletal abnormalities. Certain surgeries for example work best when performed on a child that has already undergone puberty. The chronological timing of puberty and the adolescent growth spurt however vary greatly between individuals and are affected by genetic and environmental factors. Furthermore, puberty typically occurs 2 years earlier in girls than in boys.

The invention provides a method of identifying the skeletal maturity stage of a mammal by determining the level of IGF-1 in a biological sample obtained from the mammal. The biological sample is desirably blood, serum, saliva, or urine. Based on the levels of IGF-1 in the biological sample, the skeletal maturity of the mammal can be determined. The timing of peak mandibular growth can be correlated with six cervical vertebral stages. In cervical stage 1 (CS1), the second, third, and fourth cervical vertebra (C2, C3, and C4) have flat inferior borders, and C3, and C4 are trapezoid in shape. Peak mandibular growth occurs on average two years after this stage. In CS2, C2 is characterized by a concave inferior border, and C3 and C4 have trapezoid bodies. Peak mandibular growth occurs on average one year after this stage. In CS3, C2 and C3 have concave inferior borders, while C4 is flat. In this stage, C3 and C4 are horizontally rectangular in shape. Peak mandibular growth occurs the following year. In CS4, the inferior borders of C2, C3, and C4 are concave and C3, and 4 are still horizontally rectangular in shape. Peak mandibular growth occurs within a year or two before this stage. In CS5, the inferior borders of C2, 3, and 4 are still concave and the body of C3 or C4 is square in shape with the other one remaining horizontally rectangular, if not square. Peak mandibular growth ends at least one year before this stage. In CS6, the inferior borders of C2, 3, and 4 are concave, and the body of C3 or C4 is vertically rectangular. Peak mandibular growth ends at least two years prior to this stage.

In one method, a biological sample is obtained from a mammal and IGF-1 levels are measured. IGF-1 levels ranging between 120 and 280 ug/L, 130 and 270 ug/L, 140 and 260 ug/L, 150 and 250 ug/L, or 160 and 250 ug/L indicate that a mammal in prepubertal stage is at CS1 or CS2, levels ranging between 250 and 330 ug/L, 260 and 330 ug/L, 270 and 320 ug/L, or 280 and 320 ug/L indicate that a mammal in early pubertal stage is at CS3, levels ranging between 310 and 400 ug/L, 320 and 390 ug/L, 320 and 380 ug/L, or 330 and 380 ug/L indicate that a mammal in pubertal stage is at CS4, levels ranging between 350 and 600 ug/L, 360 and 550 ug/L, 380 and 500 ug/L, 390 and 500 ug/L, or 400 and 500 ug/L indicate that a mammal in late pubertal stage is at CS5, and levels ranging between 200 and 450 ug/L, 200 and 400 ug/L, or 250 ug/L indicate that a mammal in post pubertal stage is at CS6. Pubertal stage is determined based on chronological age or sexual characteristics of the mammal.

Using the information obtained as described above, the proper timing for a medical or dental procedure is determined. For example, a mammal that is identified as being at CS6 and has IGF-1 that are less than 300 ug/L, 290 ug/L, 280 ug/L, 270 ug/L, 260 ug/L, 250 ug/L, 230 ug/L, or 200 ug/L, is one that has gone through its growth spurt. Since there should not be any residual bone growth, this mammal is a candidate for a medical procedure where residual bone growth is undesirable. Alternatively, a mammal whose IGF-1 levels range between 260 and 500 ug/L and who is identified as being at CS1, CS2, CS3, CS4, or CS5 is a candidate for a medical procedure that involves or requires residual bone growth. Alternatively, a mammal having IGF-1 levels below 280 ug/L and identified as being at CS6 is a candidate for a medical procedure that does not involve residual bone growth.

Alternatively, samples are collected periodically from a mammal (e.g., every two years, every year, every six months, or every three months) and the levels of IGF-1 in the samples are measured over time. The skeletal maturity stage of the mammal is determined at each time point. An increase in IGP-1 levels over time indicates that the mammal is between cervical stage CS1 and CS5 and a reduction of IGF-1 levels over time indicates that the mammal is between CS5 and CS6. For example, a mammal whose IGF-1 levels are increasing over time and is identified as being at CS1, CS2, CS3, CS4, or CS5 is selected as a candidate for a medical procedure that involves residual bone growth. Desirably, the levels of IGF-1 are greater than 150 ug/L, 200 ug/L, or 250 ug/L. For procedures in which complete bone growth should be complete (i.e., no residual bone growth expected), a candidate mammal is one having been identified as being at CS6 and whose IGF-1 levels are less than 300 ug/L, 290 ug/L, 280 ug/L, or 250 ug/L. Samples may be collected at anytime i.e., pre-pubertal stage, pubertal stage, late pubertal stage, and post-pubertal stage.

### IGF-1 Measurements

IGF-1 plays a key role in systemic and local regulation of both pre and postnatal longitudinal bone growth. Growth hormone (GH) and IGF-I, but not IGF-II, are important for the pubertal growth spurt. IGF-I actions are GH-dependent during prepubertal growth, but are GH-dependent and GH-independent during pubertal growth since IGF-1 can be directly stimulated by androgens during this period. Increasing IGF-1 concentrations in vitro significantly increases longitudinal bone growth in both the condyle and the femoral head without affecting their histological organization. The condyle is also more responsive and sensitive to IGF-1 than the femoral head. In vivo, the local injection of IGF-1 into the articular capsule of mature rat condyles reactivates and stimulates the process of endochondral bone formation. A longitudinal rabbit study showed that mean IGF-1 levels showed a progressive increase from 2 weeks of age, up to their peak readings at 12 weeks in males and 14 weeks in females, followed by a progressive decrease to prepubertal levels. Positive correlation coefficients were found between serum IGF-1 levels and growth increments for each of the different growth parameters measured in individual animals between 8 and 16 weeks of age (Masoud et al., Normal and Abnormal Bone Growth: Basic and Clinical Research. 233-243, 1985).

In a cross sectional study that included 1030 healthy children, adolescents, and adults, IGF-1 levels were related to age, sex, sexual development (breast buds, and testicular volume), and body mass index (Juul et al., J. Clin. Endocrinol. Metab. 78:744-52, 1994).

In the methods described herein, IGF-1 is measured in any biological sample obtained from a mammal including blood, serum, urine, and saliva as described, for example by Hizuka et al., J. Clin. Endocrinol. Metab. 64:1309-12, 1987; Costigan et al., Clin. Endocrinol. Metab. 66:1014-8, 1988; Teale J. Ann. Clin. Chem. 23:413-24, 1986; and Juul et al., J. Clin. Endocrinol. Metab. 78:744-52, 1994, all of which are hereby incorporated by reference. For example, IGF-1 levels may be determined by means of a blood spot measurement, which is a minimally invasive technique and is described, for example, in Diamandi et al., J Clin Endocrinol Metab. 83:2296-301, 1998; Wu et al., Ann. Clin. Lab. Sci. 32:287-91, 2002; Schutt et al., Growth Horm. IGF Res. 13:75-80, 2003. Using this technique, samples are stable at room temperature for up to 2 weeks.

### Medical Procedures

Determining skeletal maturity is instrumental for the proper design and timing of various medical procedures. Certain treatments for example require a certain amount of bone growth in order to be efficacious. In particular, certain dental procedures are optimal when performed on patients in whom there is residual facial bone growth (e.g., mandibular growth).

Other medical procedures are only beneficial when performed when there is no residual bone growth for example in patients, in whom a growth spurt has already occurred. Examples of such procedures are tooth implantation and orthodontic treatment. The methods described herein are useful, for example, to determine whether vertical facial growth is complete before implant placement in young adults since hand x-rays are not accurate in determining the completion of vertical facial growth. Any residual vertical growth would lead to submergence of the implant and a compromised esthetic and occlusal result. For orthodontic treatment, growth of the mandible, which mainly occurs at the condyles, is of major concern. Condylar growth however differs from growth at the epiphyseal growth plates in various aspects. For example, the mandibular condyle is a secondary cartilage, which is initially formed by intramembraneous bone formation and is later exposed to functional and developmental conditions such as compressive forces at its articular contact resulting in hypoxic and anoxic conditions. These conditions contribute to the formation of the fibrous connective tissue around the articular surface of the condyle from the original periosteum and to the metaplastic development of chondroblasts from undifferentiated connective tissue stem cells deep to the periosteum. Furthermore, the linear columns of dividing chondroblasts that commit long bones to a unidirectional mode of growth are also absent. This has the functional importance of giving the condylar cartilage the multidirectional growth capacity needed to provide the best anatomical placement of the mandibular arch, and allowing it to adapt to dental and skeletal changes during growth. Proliferating chondrocytes in the condyle also express both type I and II collagens in contrast to the growth plates of long bones which only express type II collagen during the proliferation stage. Determining the skeletal maturity before performing orthodontic treatment is pivotal in properly timing the procedure and getting optimal results.

Optionally, viewing the cervical vertebra in lateral cephalometric radiographs may also be used. Alternatively, a hand-wrist radiograph is also performed. Skeletal maturity is determined using the Greulich and Pyle technique or the Fishman technique. The Fishman technique has shown that mandibular growth lags behind statural growth with peak mandibular growth velocity occurring in the later stages of puberty. The pubertal growth curves are asymmetric with rapid acceleration up to peak growth velocity and a more gradual decline from the peak. This was more significant for mandibular growth than maxillary or statural growth.

### Materials and Methods

The sample consisted of 100 children from the orthodontic practice at KAAU in Jeddah, Saudi Arabia, as well as a private orthodontic practice in the same city. The two centers were selected to geographically and socioeconomically represent the city of Jeddah. Although the Basic Mediterranean Caucasian race was the most represented, the majority of the subjects could not be given a specific racial designation. Inclusion criteria were: 1) patients who were either to begin orthodontic treatment, were in treatment, or were in post treatment follow up; 2) Boys and girls between the ages of 5 and 25, 3) No systemic illness, growth abnormality, or bleeding disorder.

The purpose of the study and degree of involvement that was requested was explained to both the patients and parents. If they were interested in participating, they were asked to sign either an English or Arabic consent form based on the language they felt more comfortable with. KAAU offers free orthodontic treatment, and it was explained to them verbally, and in the consent forms that their participation would not affect the service that they receive.

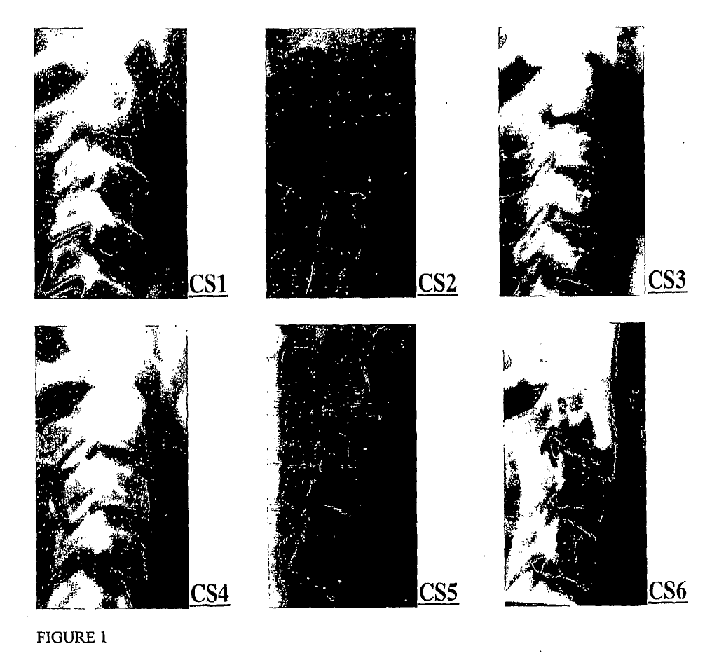

In addition to the lateral cephaplometric radiographs the patients had to fill out a questionnaire that included questions about age and pubertal status, as well as any history of blood disorders. Assistance was available to clarify any questions or concerns during this process. After that, a blood spot sample was taken, and the patient's height and weight were recorded. The blood spot samples were collected using kits donated by ZRT laboratories (Beaverton, Oreg.). The samples were stored in sealed plastic bags in a freezer for no more than 4 months. The samples were then shipped to ZRT laboratories and where assayed using an RIA technique. The examiners were blinded when staging the x-rays so that they had no information regarding the patients' IGF-1 levels. The CS technique as described by Bacetti et al. (Sem in Orthod. 11:119-129, 2005) was used to stage the cervical vertebra. Curvatures were called when the depth of the curvature was 1 mm or greater, and a mm ruler was used to measure the posterior and inferior borders to determine vertebral shape. X-rays were staged by two examiners. There was no statistical significance between the examiners. The ones that were not agreed upon were reviewed again and remeasured by both examiners until agreed on. FIG. 1 shows x-rays that were considered as reference examples for the 6 stages.

The obtained data was analyzed using the following statistics. The Kappa coefficient was calculated to determine intra and inter examiner reliability. ANOVA and a POST-HOC (LSD) tests were used to determine the differences between the mean IGF-1 levels corresponding to the skeletal maturation stages as determined by the x-rays. Linear correlations were done to determine the trends that IGF-1 levels followed at different skeletal maturation stages

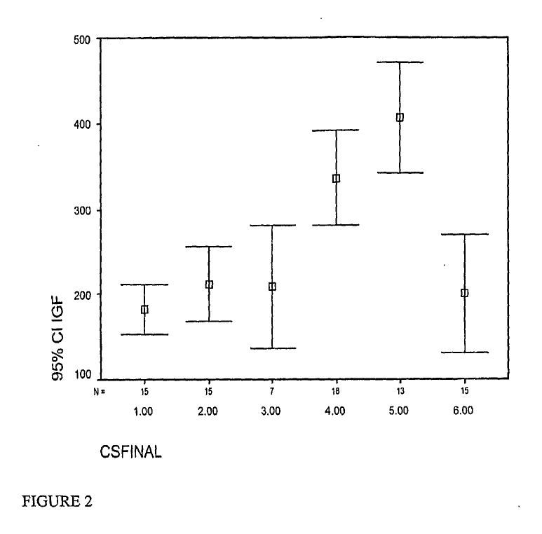

Two subjects were eliminated from the study because their actual ages was greater than their reported ages. This resulted in them not satisfying our inclusion criteria. One subject was excluded because her height was well below the lower five percentile of the population, and one subject was eliminated because the lateral cephalometric radiograph did not show the cervical vertebra. Interexaminer reliability was measured at 0.794, while intraexaminer reliability was measured at 0.824. Both were significant at the 0.001 level. FIG. 2 displays mean IFG-1 levels and 95% confidence intervals plotted against the cervical stages. Table 1 displays descriptive IGF-1 statistics for each of the cervical stages.

TABLE I
Descriptive IGF-1 statistics for each cervical stage

| | | | | | 95% | | | |
|---|---|---|---|---|---|---|---|---|
| | | | | | Confidence | | | |
| | | | | | Interval for | | | |
| Cervical | | | Std. | | Mean | Upper | | |
| Stage | N | Mean IGF | Deviation | Std. Error | Lower Bound | Bound | Minimum | Maximum |
| 1 | 15 | 182.0074 | 52.3509 | 13.5169 | 153.0164 | 210.9984 | 56.61 | 261 |
| 2 | 15 | 212.1 | 79.0111 | 20.4006 | 168.3451 | 255.8549 | 73.5 | 336 |
| 3 | 7 | 208.6849 | 78.1142 | 29.5244 | 136.4413 | 280.9284 | 106.44 | 310 |
| 4 | 18 | 335.8937 | 110.8929 | 26.1377 | 280.7479 | 391.0394 | 126.5 | 540 |
| 5 | 13 | 406.8225 | 105.4005 | 29.2328 | 343.1296 | 470.5153 | 235 | 609 |
| 6 | 15 | 199.7583 | 126.0184 | 32.5378 | 129.9717 | 269.545 | 52.7 | 416 |
| Total | 83 | 261.4887 | 126.3624 | 13.8701 | 233.8967 | 289.0806 | 52.7 | 609 |

One way ANOVA Post Hoc testing showed that IGF-1 levels at CS5 were statistically significantly greater than at stages CS1, CS2, CS3, and CS6 with a P value <0.001, and greater than IGF-1 levels at CS4 with a P value <0.05. IGF-1 levels at CS4 were greater than at CS1, CS2, and CS6 with a P value <0.001, but were greater than the

TABLE II
P values of between and within cervical
stage group comparisons, 1-way ANOVA

| | Sum of Squares | df | Mean Square | F | Sig. |
|---|---|---|---|---|---|
| Between | 582259.617 | 5 | 116451.923 | 12.333 | *** |
| Groups | | | | | |
| Within | 727071.152 | 77 | 9442.482 | | NS |
| Groups | | | | | |
| Total | 1309330.768 | 82 | | | |

* P < .05;
** P < .01;
*** P < .001;
NS, not significant.
levels at CS3 with a P value < 0.01 (Tables II and III).

Pearson's linear correlation from CS1 to CS5 exhibited a correlation coefficient of +0.68, while the correlation coefficient from CS5 to CS6 was −0.676. Both were significant at the 0.001 level. Within CS6, IGF-1 levels correlated negatively with chronological age and the number of months reported to have passed since the onset of puberty with correlation coefficients of −0.531, and −0.522 respectively. These were significant at the 0.05 level.

The data shows that IGF-1 levels start low in the prepubertal stages. There was a sharp increase in IGF-1 levels from CS3 to the peak levels at CS5. Between CS5 and CS6 IGF-1 levels gradually declined. Mean IGF-1 levels at CS4 and 5 were significantly higher than the rest of the stages, indicating that IGF-1 levels peak in late puberty. This study shows that there is a good correlation between cervical stages and IGF-1 levels with the correlation being positive from CS1 to CS5, and negative from CS5 to CS6. Baccetti et al. have demonstrated a correlation between mandibular growth and the 6 cervical stages, and our results are consistent with their findings in that IGF-1 levels peaked after stage CS3. A wide degree of individual variation was observed in IGF-1 levels within each cervical stage. However, there is a great deal of individual variation in the yearly increment of mandibular growth. For example, there is a substantial variation in condylar growth with individuals having little or negative growth while others showing more than 5 mm of growth per year. Interestingly, IGF-1 levels were still relatively high in a large number of individuals who were at CS6 and had supposedly completed their

TABLE III
P values for One-way ANOVA Post Hoc (LSD) comparisons
between IGF-1 values for the 5 cervical stages.

| | | | | | 95% | |
|---|---|---|---|---|---|---|
| | | | | | Confidence | |
| | | Mean | | | Interval | |
| (I) | (J) | Difference | | | Lower | Upper |
| CS | CS | (I − J) | Std. Error | Sig. | Bound | Bound |
| 1 | 2 | −30.0926 | 35.4824 | NS | −100.747 | 40.5618 |
| | 3 | −26.6775 | 44.4795 | NS | −115.2474 | 61.8925 |
| | 4 | −153.8863 | 33.9718 | *** | −221.5327 | −86.2398 |
| | 5 | −224.8151(*) | 36.8218 | *** | −298.1366 | −151.4935 |
| | 6 | −17.7509 | 35.4824 | NS | −88.4053 | 52.9035 |
| 2 | 1 | 30.0926 | 35.4824 | NS | −40.5618 | 100.747 |
| | 3 | 3.4151 | 44.4795 | NS | −85.1548 | 91.9851 |
| | 4 | −123.7937(*) | 33.9718 | *** | −191.4401 | −56.1472 |
| | 5 | −194.7225(*) | 36.8218 | *** | −268.044 | −121.4009 |
| | 6 | 12.3417 | 35.4824 | *** | −58.3127 | 82.9961 |
| 3 | 1 | 26.6775 | 44.4795 | NS | −61.8925 | 115.2474 |
| | 2 | −3.4151 | 44.4795 | NS | −91.9851 | 85.1548 |
| | 4 | −127.2088(*) | 43.284 | ** | −213.3984 | −41.0193 |
| | 5 | −198.1376(*) | 45.5551 | *** | −288.8495 | −107.4257 |
| | 6 | 8.9265 | 44.4795 | NS | −79.6435 | 97.4965 |
| 4 | 1 | 153.8863(*) | 33.9718 | *** | 86.2398 | 221.5327 |
| | 2 | 123.7937(*) | 33.9718 | *** | 56.1472 | 191.4401 |
| | 3 | 127.2088(*) | 43.284 | ** | 41.0193 | 213.3984 |
| | 5 | −70.9288(*) | 35.3684 | * | −141.3564 | −0.5012 |
| | 6 | 136.1353(*) | 33.9718 | *** | 68.4889 | 203.7818 |
| 5 | 1 | 224.8151(*) | 36.8218 | *** | 151.4935 | 298.1366 |
| | 2 | 194.7225(*) | 36.8218 | *** | 121.4009 | 268.044 |
| | 3 | 198.1376(*) | 45.5551 | *** | 107.4257 | 288.8495 |
| | 4 | 70.9288(*) | 35.3684 | * | 0.5012 | 141.3564 |
| | 6 | 207.0641(*) | 36.8218 | *** | 133.7426 | 280.3857 |
| 6 | 1 | 17.7509 | 35.4824 | NS | −52.9035 | 88.4053 |
| | 2 | −12.3417 | 35.4824 | NS | −82.9961 | 58.3127 |
| | 3 | −8.9265 | 44.4795 | NS | −97.4965 | 79.6435 |
| | 4 | −136.1353(*) | 33.9718 | *** | −203.7818 | −68.4889 |
| | 5 | −207.0641(*) | 36.8218 | *** | −280.3857 | −133.7426 |

* P < .05;
** P < .01;
*** P < .001;
NS, not significant.

growth, which is consistent with the fact that the mandibular condyle is more sensitive to IGF-1 than long bones and the fact that adults with acromegaly continue to experience mandibular growth well after their statural growth is complete. Our results therefore indicate that IGF-1 levels is a good indicator of residual mandibular growth.

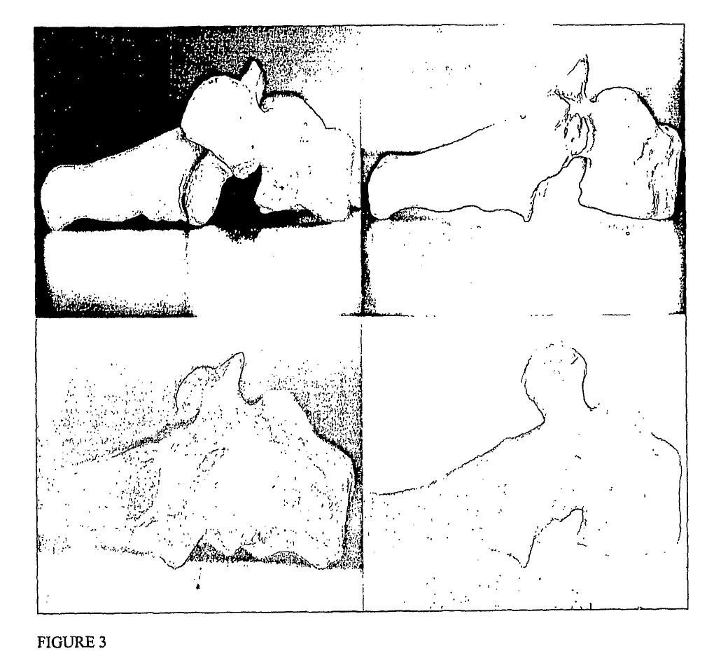

Several problems arise when using the cervical stages to assess skeletal maturity. One is that many vertebra had reached the shape of CS4 or CS5 without an apparent curvature on their inferior borders. Cervical vertebra were obtained from cadavers and x-rays were taken at different angulations. Laterally tipping a mature vertebra with an obvious curvature about 30 degrees was sufficient to make the curvature disappear on an x-ray, which may explain the absence of curvatures on the inferior borders of otherwise mature looking vertebra (FIG. 3). Even though samples were obtained from various patients in the average age of CS3 occurrence were, only 7 patients were actually in CS3. In some subjects, CS4 was detected even before CS3.

Based on the above results, assessing IGF-1 levels is an accurate means of determining skeletal maturity.

What is claimed is:

1. A method of determining skeletal maturity in a subject, comprising measuring the level of IGF-1 in a tissue or fluid sample, wherein an elevated level of IGF-1 post-puberty indicates that said subject has not reached skeletal maturity.

2. The method of claim 1, wherein said level of IGF-1 post-puberty exceeds 250 μg/L.

3. The method of claim 1, wherein said subject is suffering from or at risk of developing a mandibular or facial growth abnormality.

4. The method of claim 1, wherein said method does not comprise x-ray examination of a cervical vertebra.

5. A method of identifying the skeletal maturity stage of a mammal, comprising the steps of: determining the level of IGF-1 in said sample, wherein levels ranging between 140 and 260 ug/L indicates that a mammal in prepubertal stage is at cervical stage (CS) 1 or CS2, levels ranging between 270 and 320 ug/L indicates that a mammal in early pubertal stage is at CS3, levels ranging between 320 and 380 ug/L indicates that a mammal in pubertal stage is at CS4, levels ranging between 380 and 500 ug/L indicates that a mammal in late pubertal stage is at CS5, and levels ranging between 200 and 400 ug/L indicates that a mammal in post pubertal stage is at CS6

6. The method of claim 5, wherein a mammal having IGF-1 levels ranging between 260 and 500 ug/L and identified as being at CS1, CS2, CS3, CS4, or CS5 is selected as a candidate for a medical procedure that involves residual bone growth in said mammal.

7. The method of claim 5, wherein a mammal having IGF-1 levels below 280 ug/L and identified as being at CS6 is selected as a candidate for a medical procedure that does not involve residual bone growth.

8. The method of claim 6 or 7, wherein said bone growth is facial bone growth.

9. The method of claim 8, wherein said facial bone growth is mandibullar growth.

10. The method of claim 6 or 7, wherein said medical procedure is a dental procedure.

11. The method of claim 10, wherein said dental procedure is an orthodontic procedure.

12. The method of claim 10, wherein said dental procedure is a tooth implant.

13. The method of claim 1, wherein said biological sample is serum, blood, or saliva.

14. The method of claim 13, wherein said blood is obtained by blood spotting.

15. The method of claim 5, further comprising obtaining a hand-wrist radiograph from said mammal.

16. The method of claim 15, wherein said hand wrist radiograph is evaluated using the Greulich and Pyle technique or the Fishman technique.

17. The method of claim 5, wherein said mammal is a human.

18. The method of claim 5, wherein pre-pubertal stage, pubertal stage, late pubertal stage, and post-pubertal stage are identified by physical characteristic of said mammal.

19. The method of claim 18, wherein said physical characteristic is a sexual characteristic.

20. The method of claim 5, wherein said pre-pubertal stage, pubertal stage, late pubertal stage, and post-pubertal stage are identified by the chronological age of said mammal.

21. A method of identifying the skeletal maturity stage of a mammal, comprising the steps of:
(a) providing a biological sample from a mammal;
(b) determining the level of IGF-1 in said sample; and
(c) repeating steps (a) and (b) at one or more time intervals, wherein an increase in IGF-1 levels over time indicates that said mammal is between cervical stage CS1 and CS5 and a reduction of IGF-1 levels over time indicates that said mammal is between CS5 and CS6.

22. The method of claim 21, wherein said repeating is performed approximately every year from the time said mammal is in prepubertal stage.

23. The method of claim 22, wherein said repeating is performed approximately every six months.

24. The method of claim 21, wherein a mammal whose IGF-1 levels are increasing over time and is identified as being at CS1, CS2, CS3, CS4, or CS5 is selected as a candidate for a medical procedure that involves residual bone growth in said mammal.

25. The method of claim 21, wherein a mammal identified as being at CS6 and whose IGF-1 levels are less than 280 ug/L is selected as a candidate for a medical procedure that does not involve residual bone growth.

26. The method of claim 24 or 25, wherein said bone growth is facial bone growth.

27. The method of claim 26, wherein said facial bone growth is mandibullar growth.

28. The method of claim 24 or 25, wherein said medical procedure is a dental procedure.

29. The method of claim 28, wherein said dental procedure is an orthodontic procedure.

30. The method of claim 28, wherein said dental procedure is a tooth implant.

31. The method of claim 21, wherein said biological sample is serum, blood, or saliva.

32. The method of claim 31, wherein said blood is obtained by blood spotting.

33. The method of claim 21, further comprising obtaining a hand-wrist radiograph from said mammal.

34. The method of claim 33, wherein said hand wrist radiograph is evaluated using the Greulich and Pyle technique or the Fishman technique.

35. The method of claim 21, wherein said mammal is a human.

36. The method of claim 21, wherein pre-pubertal stage, pubertal stage, late pubertal stage, and post-pubertal stage are identified by physical characteristic of said mammal.

37. The method of claim 36, wherein said physical characteristic is a sexual characteristic.

38. The method of claim 21, wherein said pre-pubertal stage, pubertal stage, late pubertal stage, and post-pubertal stage are identified by the chronological age of said mammal.
